SEQUENCE LISTING (1 sequence)

SEQ ID NO: 1 - protein, 7 aa, Artificial Sequence
  Angiotensin(1-7)
DRVYIHP

(19) United States
(12) Patent Application Publication
(10) Pub. No.: US 2019/0328828 A1
(43) Pub. Date: Oct. 31, 2019

(54) PHARMACEUTICAL COMPOSITION, PROCESS FOR PRODUCING THE SAME, USE OF A PEPTIDE, USE OF A PHARMACEUTICAL COMPOSITION AND METHOD FOR TREATING DISEASES ASSOCIATED WITH INTRAOCULAR HYPERTENSION OR GLAUCOMA

(71) Applicant: UNIAO QUIMICA FARMACEUTICA NACIONAL S/A, Embu-Guacu (BR)

(72) Inventors: Miquel Giudicissi FILHO, Sao Paulo (BR); Jose Eduardo Sacconi NUNES, Aguas Claras (BR); Reiner Ludwig GENTZ, Belo Horizonte (BR)

(21) Appl. No.: 16/472,115

(22) PCT Filed: Jan. 8, 2018
(86) PCT No.: PCT/BR2018/050004

§ 371 Date: Jun. 20, 2019

(30) Foreign Application Priority Data
Jan. 6, 2017 (BR) 102017000331-0

(51) Int. Cl. A61K 38/08; A61K 47/40; A61K 9/127; A61K 9/50; A61K 9/19; A61K 9/00; A61P 27/06

(52) U.S. Cl. CPC A61K 38/085; A61K 47/40; A61K 9/1271; A61K 9/5021; A61P 27/06; A61K 9/0019; A61K 9/0053; A61K 9/0048; A61K 9/19

(57) ABSTRACT

The present invention describes a pharmaceutical composition of biologically active peptides, associated with a controlled release system using cyclodextrins or derivatives thereof, liposomes and biodegradable polymers and/or mixtures of said systems for increasing bioavailability, duration and intensity of the biological effects of the peptide. Specifically, the present invention comprises a pharmaceutical composition, a process for preparing same, and the use of a peptide in said composition for preparing medication for intraocular hypertension or glaucoma. The present invention falls within the field of Medical Science, more specifically of preparations for medical purposes, and even more specifically of medicinal preparations containing peptides.

### FIELD OF THE INVENTION

The present invention describes a pharmaceutical composition of biologically active peptides, bound to a controlled release system using cyclodextrins or derivatives thereof, liposomes and biodegradable polymers. The present invention falls in the field of Medical Science, more specifically preparations for medical purposes, still more specifically medicinal preparations containing peptides.

### BACKGROUND OF THE INVENTION

Glaucoma is a group of diseases of heterogeneous eye from the point of view of the pathogenesis and its clinical expression. It is characterized by progressive damage to the optic nerve, ultimately leading to irreversible blindness. Glaucoma is estimated to affect about 70 million people around the world (Thylefors B, 1996; Quadley H. A., 1996) and, with increasing life expectancy and consequent growth of the elderly population, this number is expected to increase (Friedman D. S., 2004).

Glaucoma is often classified as a primary open-angle, primary closed-angle, secondary, and congenital, although other types, such as normal pressure glaucoma, exist. The intraocular pressure in a healthy individual is about 15 mmHg (Milar C., 1995). In many cases of glaucoma, loss of vision is related to high intraocular pressures with subsequent damage to the optic nerve (Hollows F. C., 1966).

The intraocular pressure is needed to inflate the eye, maintaining a suitable shape and optical properties of the eyeball. This pressure is generated by the difference between the production and drainage of the aqueous humor. The increase in resistance during drainage is what generates an increase in intraocular pressure and has been considered a basic principle in the pathophysiology of glaucoma. The aqueous humor is a transparent liquid filling and helps to give shape to anterior and posterior chambers of the eye. The lenses and cornea must stay translucent in order to allow transmission of light, and therefore cannot receive vascularization. The aqueous humor is analogous to a blood substitute for these avascularized structures and provides nutrition, removes waste products from metabolism, carries neurotransmitter, stabilizes the ocular structure and contributes for regulating homeostasis of such eye tissues (Sires B., 1997).

Main eye structures related to the dynamics of aqueous humor are the ciliary body (the production site of aqueous humor), the trabecular meshwork and the uveoscleral pathway (major responsible for draining the aqueous humor). Aqueous humor production is an active metabolic process and involves three different mechanisms: diffusion, ultrafiltration and active secretion (Millar C., 1995).

Diffusion occurs when solutes, especially lipid-soluble substances, are carried through the membrane of the tissues between the capillaries and the posterior chamber, proportional to a concentration gradient across the membrane (Civan M. M., 2004). Ultrafiltration corresponds to the flow of water and water-soluble substances, limited by size and load, through the ciliary capillary endothelium for the ciliary stroma, in response to an osmotic gradient and hydrostatic pressure (Smith R. S., 1973; Uusitalo R., 1973). The active secretion is believed to be the greatest contributor to aqueous humor formation, responsible for approximately 80-90% of the total (Gabelt B. T., 2003; Mark H. H., 2009). The main tissue responsible for active secretion is the non-pigmented epithelium of the ciliary body. Active secretion occurs through selective transcellular movement of cations, anions, and other molecules through a concentration gradient in the aqueous-blood barrier. This occurs by means of transport proteins, such as aquaporins, which obtains energy for this process through hydrolysis of adenosine triphosphate (ATP) (Yamaguchi Y., 2006).

Another enzyme to be considered in the process of producing aqueous humor is carbonic anhydrase, found in the pigmented and non-pigmented ciliary epithelia (Dobbs P. C., 1979), which mediates the transport of bicarbonate across the ciliary epithelium through reversible hydration of CO₂ to form HCO₃⁻ and protons through the following reaction: CO₂+H₂O⇄H₂(CO)₃⇄HCO₃⁻+H⁺ (Wistrand P. J., 1951). Bicarbonate formation influences the transport of fluids, what affects the concentration of sodium ions, possibly by regulating the pH for optimizing the active ion transport.

The movement of electrolytes through the ciliary epithelium is regulated by electrochemical gradients and, while in a liquid balance there is a secretion direction across the epithelium (Gabelt B. T., 2003), oncotic and hydrostatic forces favor the resorption of aqueous humor (Bill A., 1973). Particularly some studies (Moses R., 1981; Kiel J. W., 2011) show that when intraocular pressure is close to blood pressure of ciliary artery, the formation of aqueous humor decreases rapidly. Therefore, by regulating the arterial pressure locally, it is possible to obtain a regulation of aqueous humor production.

The renin-angiotensin system (RAS) is responsible for regulating arterial pressure, cardiovascular homeostasis and hydroelectrolytic balance, in both physiological and pathological conditions (Krieger, E. M.; Santos, R. A. S. Angiotensinas—aspectos fisiológicos. Hipertensão, 1: 7-10, 1998). Angiotensin II (Ang II) is the main effector peptide of RAS, having vasopressor actions, stimulating the synthesis of adrenal, proliferative (fibroblasts, vascular smooth muscle) and hypertrophic (cardiac myocytes) steroids. Its formation pathway involves the production of angiotensinogen by the liver and the production of renin in the juxtaglomerular apparatus. These substances are delivered into the bloodstream, where the angiotensinogen is hydrolyzed by renin, forming angiotensin I (Ang I), which in the lung will undergo to action of the Angiotensin-converting enzyme (ACE) and will produce Ang II. In turn, this will exert its actions on target organs away from the site of its production (Krieger, E. M.; Santos, R. A. S. Angiotensinas—aspectos fisiológicos. Hipertensão, 1: 7-10, 1998).

Recently, it has been found that in addition to the system generating circulating Ang II, different tissues contain independent RAS that generate Ang II, apparently for local action. Components of the tissue RAS are found in the walls of the blood vessels, in uterus, in exocrine portion of the pancreas, eyes, heart, adrenal cortex, testis, ovaries, anterior and intermediate lobes of the pituitary gland, pineal gland and brain. The functions of such tissue RAS are not very well understood. (Ardaillou, R.; Michel, J. B. The relative roles of circulating and tissue renin-angiotensin systems. Nephrol. Dial. Transplant., 14:283-286, 1999). The local actions of RAS may occur at the cell level producing peptides (intracrine and autocrine functions), on adjacent cells (paracrine function) or at locations remote from the production region (endocrine function).

ACE inhibitors have been investigated as a new class of drugs in the treatment of glaucoma. It has been shown that they can reduce intraocular pressure (10P) in patients with ocular hypertension or glaucoma (Constad et al. 1988). In another study, it was noted that enalaprilat lowered 10P in humans, but this effect was blocked by indomethacin, suggesting the participation of prostaglandins in the hypotensive mechanism of Conversion Enzyme inhibitors (Lotti and Pawlowski 1990). These inhibitors also inhibit kininase II and therefore prevent bradykinin metabolism, increasing the production of prostaglandins, which act by increasing the uveoscleral outflow (Crawford and Kaufmann 1987). Shah et al. (2000) noted reduction of 10P in rabbits with ocular hypertension subjected to the topical use of enalaprilat, ramiprilat and fosinopril.

In addition to hypotensive action, there are signs that ACE inhibitors can inhibit apoptosis of nerve cells. In fact, two randomized clinical trials showed a significative inverse relationship between use of anti-hypertensive drugs and risk of dementia (Forette et al, 2002; Tzourio et al 2003). Study of Systolic Hypertension in Europe (SYST-EUR) has noted a 55% reduction of the risk of dementia in patients treated chronically with enalapril (Forette et al. 2002), while PROGRESS (acronym in English for “Protection of Perindopril against recurrent Stroke study”) has showed a 34% reduction in the risk of developing dementia with perindopril (Tzourio et al. 2003). Bradykinin, increased in patients receiving conversion enzyme inhibitors, it is a protector agent against the neurotoxic action of glutamate in neuron culture (Yasuochi et al 2004). This likely occurs due to the increase in superoxide dismutase activity, which modulates the production of nitric oxide and inactivates the reactive species of oxygen and other pro-oxidative mechanisms (Ehring et al. 1994). These findings have a probable relationship with the fact that patients with normal pressure glaucoma have increased sensitivity to bradykinin, what in turn can indicate that those patients have lowered levels of bradykinin (Hirooka et al. 2002). Recently, it was noted that a bradykinin-receptor 2 agonist produced hypotensive eye effect in monkeys (Sharif 2015).

Other studies have evaluated the effect of losartan, an angiotensin II receptor inhibitor, in intraocular pressure of normal subjects, arterial hypertensive without glaucoma and patients with glaucoma without arterial hypertension. In all groups there was a reduction in intraocular pressure, but the reduction of arterial pressure occurred only in group with arterial hypertension. There was an increase in the ease of outflow of aqueous humor in all patients, suggesting that this is the mechanism of action of losartan, and not an effect mediated by reducing arterial pressure. (Costagliola et al 2000). These findings were confirmed by Hashizume et al. (2005). Recently, White et al (2015) noted that, in a rat retina explant model, another angiotensin II inhibitor (irbesartan) doubled the survival of ganglion cells, while angiotensin II reduced the survival by 40%, likely through the activation of At1R receptors, associated with an NADPH-dependent pathway resulting in superoxide production.

On the other hand, more recent researches arose the possibility of an ACE activator (diminazene aceturate-DIZE) acting on the intraocular pressure of glaucomatous rats. In fact, DIZE produced an increase in outflow of aqueous humor and a significant reduction in intraocular pressure in both forms of eye drops and after systemic administration (Foureaux G et al 2013).

Recent observations indicate that important peripheral and central actions of the RAS may be mediated by smaller sequences of angiotensinergic peptides, including Angiotensin-Ill [Ang-(2-8)], Angiotensin-IV [Ang-(3-8)] and Angiotensin-(1-7). It can be appreciated that both Angiotensin-I [Ang-(1-10)] and Angiotensin-II [Ang-(1-8)] can undergo to biotransformation process, generating a “family” of biologically active angiotensin peptides. (Santos, R. A. S.; Campagnole-Santos, M. J.; Andrade, S. P. Angiotensin-(1-7): an update. Regulatory Peptides, 91:45-62, 2000).

Angiotensin-(1-7) is one of the peptides from the “family” of biologically active angiotensins, being formed by an independent pathway from ACE. The processing of Ang I by endopeptidases or Ang II by prolyl-peptidases or carboxy-peptidases generates heptapeptide Ang-(1-7). Once formed, Ang-(1-7) can be hydrolyzed by amino-peptidases generating Ang-(2-7) and Ang-(3-7). The hydrolysis of Ang-(1-7) by ACE originates Ang-(1-5). (Santos, R. A. S.; Campagnole-Santos, M. J.; Andrade, S. P. Angiotensin-(1-7): an update. Regulatory Peptides, 91:45-62, 2000).

Ang-(1-7) along with Ang II, are the main effectors of RAS. Two main features separate Ang-(1-7) from Ang II: the first has highly specific biological actions and formation pathway thereof is independent of ACE (Santos, R. A. S.; Campagnole-Santos, M. J.; Andrade, S. P. Angiotensin-(1-7): an update. Regulatory Peptides, 91:45-62, 2000).

Angiotensin-(1-7), (Asp-Arg-Val-Tyr-Ile-His-Pro) and its Sar1-Ang-(1-7) derivative also antagonize the pressor effects of Ang II in man (Ueda S, Masumori-Maemoto S, Ashino K, Nagahara T, Gotoh E, Umemura S, Ishii M. Angiotensin-(1-7) attenuates vasoconstriction evoked by angiotensin II but not by noradrenaline in man. Hypertension 2000; 35:998-1001) and in rats (Bovy P R, Trapani A J, McMahon E G, Palomo M. A carboxy-terminus truncated analogue of angiotensin II [Sar1] angiotensin II-(1-7)-amide, provides an entry to a new class of angiotensin II antagonists. J Med Chem. 1989; 32:520-522). The contraction produced by Ang II in isolated arteries of rabbits and humans is also reduced by angiotensin-(1-7) (Bovy P R, Trapani A J, McMahon E G, Palomo M. A carboxy-terminus truncated analogue of angiotensin II [Sar1] angiotensin II-(1-7)-amide, provides an entry to a new class of angiotensin II antagonists. J Med Chem. 1989; 32:520-522; Roks A J, Van-Geel P P, Pinto Y M, Buikema H, Henning R H, de Zeeuw D, van-Gilst W H. Angiotensin-(1-7) is a modulator of the human renin-angiotensin system. Hypertension 1999; 34(2):296-301). Recently, Holappa et al (2015) showed the presence of Ang (1-7), ACE1 and ACE2 in aqueous humor of patients with cataract, and they noted that their concentrations were higher in glaucomatous patients.

Receptors responsible for the transduction of Ang-(1-7) signal still remain undefined, and there may be several possibilities related to signal mediation. The first evidence of the existence of different receptors and/or of differentiated mechanisms of signal transduction for Ang-(1-7), is based on the opposite and/or different actions between Ang II and Ang-(1-7). Recently, heptapeptide D-[Ala 7]-Ang-(1-7) (A-779) was characterized as a potent Ang-(1-7) antagonist (Santos R A S, Campagnole-Santos M J, Baracho N C V, Fontes M A P, Silva L C S, Neves L A A, Oliveira D R, Caligiorne S M, Rodrigues A R V, Gropen Jr. C, Carvalho W S, Silva A C S, Khosla M C. Characterization of a new angiotensin antagonist selective for angiotensin-(1-7): Evidence that the actions of angiotensin-(1-7) are mediated by specific angiotensin receptors. Brain Res. Bull. 1994; 35:293-299). The results of such study indicated that this peptide is a selective Ang-(1-7) antagonist without demonstrating agonist activity in several biological preparations. This peptide has been found to be potent in antagonizing the anti-diuretic effect of Ang-(1-7) in rats with water overload. Vasodilatation produced by Ang-(1-7) in the afferent arterioles of rabbits, its pressor effect on RVLM, the vasodilatation produced in the mesenteric microcirculation in vivo, are fully blocked by A-779 administration, not being modified by Ang II antagonists. Other studies with cultures of bovine endothelial cells, coronary arteries of dogs, aorta of SHR, human epithelial fibroblasts, human heart fibroblasts and kidney cutouts have provided evidences for the existence of specific Ang-(1-7)-receptors blocked by A-779. (Santos, R A S; Campagnole-Santos, M J; Andrade, S P. Angiotensin-(1-7): an update. Regulatory Peptides, 91:45-62, 2000).

A-779 and analogs thereof, such as Sarcosine1-D-Ala 7-Ang-(1-7) (Bovy P R, Trapani A J, McMahon E G, Palomo M. A carboxy-terminus truncated analogue of angiotensin II [Sar1] angiotensin II-(1-7)-amide, provides an entry to a new class of angiotensin II antagonists. J Med Chem. 1989; 32:520-522.), and D-Pro7-Ang-(1-7) (Naves-Santos, V., Khosla, M. C., Oliveira, R. C., Campagnole-Santos, M. J., Lima, D. X., Santos, R A S. Inibição seletiva do efeito pressor central da angiotensina-(1-7) pelo seu análogo [D-Pro7]-angiotensina-(1-7). XI Reunião Annual da Federação de Sociedade de Biologic Experimental, 1996, Caxambu, MG) and other ones may serve as extremely useful tools for explaining biological effects of Ang-(1-7).

It has been showed that Ang-(1-7) acts as a counter-regulating peptide within the renin-angiotensin system, acting on multiple points (Ferrario C M, Chappell M C, Dean R H, Iyer S N. Novel angiotensin peptides regulate arterial pressure, endothelial function, and natriuresis. J Am Soc Nephrol. 1998; 9: 1716-1722. Santos, R. A S, Campagnole-Santos, M J, Andrade, S P. Angiotensin-(1-7): an update. Regulatory Peptides, 91:45-62, 2000. Henriger-Walther S, Batista E N, Walther T, Khosla M C, Santos R A S, Campagnole-Santos M J. Baroreflex improvement in SHR after ACE inhibitors involves angiotensin-(1-7). Hypertension, 37: 1309-1313, 2001).

Ang-(1-7) stimulates angiogenesis and cell proliferation (Machado, R D P, Santos, R A S, Andrade, S P. Mechanisms of angiotensin-(1-7) induced inhibition of angiogenesis. Am J Physiol, 280: 994-1000, 2001. Rodgers K, Xiong S, Felix J, Roda N, Espinoza T, Maldonado S, Dizerega G. Development of angiotensin-(1-7) as an agent to accelerate dermal repair. Wound Repair Regen, 9: 238-247, 2001) and therefore provides a potential for the treatment of injuries. Ang-(1-7) may act as an ACE-inhibitor in both the amino-terminal domain of the enzyme, in which it acts as a substrate, and in the c-terminal domain, in which acts as an inhibitor (Deddish P A, Marcic B, Jackman H L, Wang H Z, Skidgel R A, Erdös E G. N-domain-specific substrate and C-domain inhibitors of angiotensin-converting enzyme: angiotensin-(1-7) and keto-ACE. Hypertension. 1998; 31:912-917. Tom B, De Vries R, Saxena P R, Danser A H J. Bradykinin potentiation by angiotensin-(1-7) and ACE inhibitors correlates with ACE C-and N-domain blockade. Hypertension, 38: 95-99, 2001). The IC50 for inhibiting ACE by Ang-(1-7) is approximately 1 micromolar (Chappell M C, Pirro N T, Sykes A, Ferrario C M. Metabolism of angiotensin-(1-7) by angiotensin-converting enzyme. Hypertension. 1998; 31 (part 2):362-367. Paula, R D, Lima, C V, Britto, R R, Campagnole-Santos, M J, Khosla, M C, Santos, R A S. Potentiation of the hypotensive effect of bradykinin by angiotensin-(1-7)-related peptides. Peptides, v. 20, p. 493-500, 1999. Deddish P A, Marcic B, Jackman H L, Wang H Z, Skidgel R A, Erdös E G. N-domain-specific substrate and C-domain inhibitors of angiotensin-converting enzyme: angiotensin-(1-7) and keto-ACE. Hypertension, 31:912-917, 1998).

In addition to inhibiting ACE, Ang-(1-7) inhibits Ang II actions by two mechanisms: 1) competing for binding in AT1 receptors (Bovy P R, Trapani A J, McMahon E G, Palomo M. A carboxy-terminus truncated analogue of angiotensin II [Sar1] angiotensin II-(1-7)-amide, provides an entry to a new class of angiotensin II antagonists. J Med Chem. 1989; 32:520-522.—Ueda S, Masumori-Maemoto S, Ashino K, Nagahara T, Gotoh E, Umemura S, Ishii M. Angiotensin-(1-7) attenuates vasoconstriction evoked by angiotensin II but not by noradrenaline in man. Hypertension 2000; 35:998-1001. Roks A J, Van-Geel P P, Pinto Y M, Buikema H, Henning R H, deZeeuw D, van-Gilst W H. Angiotensin-(1-7) is a modulator of the human renin-angiotensin system. Hypertension 1999; 34(2):296-301. Rowe B P, Saylor D L, Speth R C, Absher D R. Angiotensin-(1-7) binding at angiotensin II receptors in the rat brain. Regul Pep. 1995; 56(2):139-146. Mahon J M, Carrr R D, Nicol A K, Hendersn I W. Angiotensin-(1-7) is an antagonist at the type 1 angiotensin II receptor. J Hypertension 1994; 12:1377-1381), and 2) changing the signalling of Ang II effects, possibly by changing the availability of intracellular calcium (Chansel D, Vandermeerch S, Andrzej O, Curat C, Ardaillou R. Effects of angiotensin IV and angiotensin-(1-7) on basal angiotensin II-stimulated cytosolic Ca+2 in mesangial cells. Eur J Pharmacol. 2001; 414:165-175). A third mechanism by which Ang-(1-7) antagonizes the harmful effects of Ang II on the cardiovascular apparatus is through enhancement of bradykinin effects (Paula, R D; Lima, C V, Khosla, M C, Santos, R A S. Angiotensin-(1-7) potentiates the hypotensive effect of bradykinin in concious rats. Hypertension, 26: 1154-1159, 1995. Li P, Chappell M C, Ferrario C M, Brosnihan K B. Angiotensin-(1-7) augments bradykinin-induced vasodilation by competing with ACE and releasing nitric oxide. Hypertension. 1997; 29 (part 2):394-400).

Bradykinin is an endogenous peptide with potent vasodilating action (Rocha e Silva, M, Beraldo, W T, Rosenfeld, G. Bradykinin, a hypotensive and smooth muscle stimulating factor releases from plasma globulin by snake venoms and by trypsin. Am. J. Physiol. 156, 261-273, 1949). Beneficial actions of bradykinin in the heart have been also described (Linz W, Wohlfart P, Scholkens B A, Malinski T, Wiemer G. Interactions among ACE, kinins and NO. Cardiovasc Res. 1999; 43:549-561). Ang-(1-7) enhances the bradykinin effects both in vessels (Paula, R. D.; Lima, C. V.; Khosla, M. C.; Santos, R. A. S. Angiotensin-(1-7) potentiates the hypotensive effect of bradykinin in concious rats. Hypertension, 26: 1154-1159, 1995. Li P, Chappell M C, Ferrario C M, Brosnihan K B. Angiotensin-(1-7) augments bradykinin-induced vasodilation by competing with ACE and releasing nitric oxide. Hypertension. 1997; 29 (part 2):394-400) and in heart (Almeida, A P, Frábregas, B C, Madureira, M M, Santos, R J S, Campagnole-Santos, M J, Santos, R A S. Angiotensin-(1-7 potentiates the coronary vasodilatory effect of bradykinin in the isolated rat heart. Brazilian Journal of Medical and Biological Research, 33: 709-713, 2000).

In U.S. Pat. No. 5,834,432, agonists of AT2 receptors were used to speed up the wound healing.

A drug can be chemically modified to change the biodistribution, pharmacokinetics and solubility properties thereof. Several methods have been used to increase the solubility and stability of drugs, among them the use of organic solvents, emulsions, liposomes, pH adjustment, chemical modifications and complexation of the drugs with a suitable encapsulating agent such as cyclodextrins, liposomes and microencapsulation in biodegradable polymers.

Cyclodextrins were first isolated in 1891 by Vilers, such as starch degradation products through the action of Bacillus macerans amylase. In 1904, Schardinger characterized them as cyclic oligosaccharides. In 1938 Frudenberg et al. reported that cyclodextrins are composed by glucose units joined by α(1-4) bound. Molecular weights of α, β and γcyclodextrins were determined by Frend et al. from 1942 to 1949. In 1948, Freudenberg et al. found that cyclodextrins have the ability to form inclusion compounds or complexes, and later, as well as French et al., they have been proposed synthesis processes of pure cyclodextrins. Cramer et al., as from 1954, have performed systematic study of cyclodextrins complexes formation with other compounds. Between 1955 and 1960, first studies were performed on the formation of inclusion complexes of cyclodextrins with drugs. These studies proceed extensively in Japan, Hungary, France, Italy, and in other countries.

Cyclodextrins are obtained by enzymatic degradation of starch. The methods comprise the following phases: production and purification of enzyme, enzymatic transformation of starch, and recovery and separation of cyclodextrins. The involved enzyme is a cyclodextrin-glycosyltransferase (CGT), obtained from several microorganisms, but mainly Bacillus macerans, B. megatherium, B. stereothermoplhilus e Klebsiella pneumoniae. (Korolkovas, A. Incusão molecular e ciclodextrinas: propriedades e aplicações terapêuticas. ENLACE Farmalab, 2/91, Ano 5, Vol. II, p. 6-15).

Cyclodextrins are cyclic oligosaccharides including six, seven, or eight glucopyranose units. Due to steric interactions, cyclodextrins form a cyclic structure in the form of a truncated cone with a non-polar internal cavity. It is chemically stable compounds that can be modified in a region-selective way. Cyclodextrins (hosts) form complexes with several hydrophobic molecules (guests) including the same in full or in part form in the cavity. Cyclodextrins have been used for solubilization and encapsulation of drugs, perfumes and flavorings as described by Szejtli, J., Chemical Reviews, (1998), 98, 1743-1753. Szejtli, J., J. Mater. Chem., (1997), 7, 575-587. According to detailed studies of toxicity, mutagenicity, teratogenicity and carcinogenicity over cyclodextrins, described in [Rajewski, R. A., Stella, V., J. Pharmaceutical Sciences, (1996), 85, 1142-1169], those have low toxicity, in particular, hydroxypropyl-(-cyclodextrins, as reported in Szejtli, J. Cyclodextrins: Properties and applications. Drug Investig., 2 (suppl. 4): 11-21, 1990. Except for high concentrations of some derivatives, causing damages to red blood cells, these products generally do not result in health risks. The use of cyclodextrins as additives in foods has already been authorized in countries such as Japan and Hungary, and for more specific applications in France and Denmark. All of such features are an increasing motivation for the discovery of new applications.

Administration of drugs in the incorporated form in a polymer matrix allows for its delivery into the organism in small and controllable daily doses, for days, months or even years.

Several polymers have already been tested in controlled release systems. Many as a function of their physical properties (Gilding, D. K. Biodegradable polymers. Biocompat. Clin. Implat. Mater. 2: 209-232, 1981). However, for use in humans, the material must be chemically inert and free of impurities.

Liposomes are lipid vesicles that include aqueous internal compartments in which molecules, e.g. drugs, may be encapsulated in order to achieve slow release of the drug after administration of the liposomes in a subject.

In state of the art, there are many patents for preparing liposomes [U.S. Pat. No. 4,552,803; Lenk; U.S. Pat. No. 4,310,506, Baldeschwieler; U.S. Pat. No. 4,235,871, Papahadjopoulos; U.S. Pat. No. 4,224,179, Schneider; U.S. Pat. No. 4,078,052, Papahadjopoulos; U.S. Pat. No. 4,394,372, Alfaiate; U.S. Pat. No. 4,308,166; Marchetti; U.S. Pat. No. 4,485,054; Mezei; and U.S. Pat. No. 4,508,703, Redziniak; Woodle and Papahadjopoulos, Methods Enzymol. 171:193-215 (1989)]. Unilamellar liposomes have a sole membrane including an aqueous volume [Huang, Biochemistry 8:334-352 (1969)] while multilamellar liposomes have several concentric membranes [Bangham et Col., J. Mol. Biol. 13:238-252 (1965).

The Bangham's procedure [J. Mol. Biol. 13:238-252 (1965)] produces “regular multilamellar liposomes” (MLVs). “Regular” MLVs can have uneven solute distribution among aqueous compartments and thus present an osmotic pressure difference among compartments. Lenk et Col. (U.S. Pat. Nos. 4,522,803; 5,030,453 e 5,169,637), Fountain et al. (Pat U.S. Pat. No. 4,588,578), Cullis et al. (U.S. Pat. No. 4,975,282) and Gregoriadis et al. (Pat. W.O. 99/65465) have discovered methods for preparing multilamellar liposomes having substantially even solute distribution among compartments. An even distribution of solute among different compartments means a greater efficacy of drug encapsulation, as well as a lower osmotic pressure difference, making these MLVs more stable than regular MLVs.

Unilamellar liposomes can be produced by sonicating MLVs [see Paphadjopoulos et al. (1968)] or by extrusion through polycarbonate membranes [Cullis et Col. (U.S. Pat. No. 5,008,050) e Loughrey et Col. (U.S. Pat. No. 5,059,421)].

The composition of the liposomes may be handled in order to give them a specificity for organs or cells. Targeting of liposomes was classified based on anatomical factors and involved mechanisms. The anatomical classification is based on selectivity level, e.g., organ-specific, cell-specific or organelle-specific. From the point of view of mechanisms, targeting can be considered as passive or active.

Passive targeting uses the natural trend of the conventional liposomes of being captured by the cells of the endobronchial-endothelial system in organs containing sinusoidal capillaries. Liposomes may be sterically stabilized (also known as “liposomes-PEG”), which are characterized by a lowered elimination rate from bloodstream [Lasic e Martin, Stealth Liposomes, CRC Press, Inc., Boca Raton, Fla. (1995)]. Liposomes-PEG have the polymer-coated surface, preferably polyethylene glycol (PEG) which is covalently conjugated to one of the phospholipids and creates a hydrophilic cloud outside the vesicle bilayer. This steric barrier delays the liposome recognition by the opsonins and allows the liposomes to remain longer in bloodstream than conventional liposomes [Lasic e Martin, Stealth Liposomes, CRC Press, Inc., Boca Raton, Fla. (1995); Woodle et Col., Biochim. Biophys. Acta 1105:193-200 (1992); Litzinger et Col., Biochim. Biophys. Acta 1190:99-107 (1994); Bedu Addo, et Col., Pharm. Res. 13:718-724 (1996)], and increase the pharmacological efficacy of encapsulated agents, as had been showed for some chemotherapeutics [Lasic e Martin, Stealth liposomes, CRC Press, Inc., Boca Raton, Fla. (1995)] e peptideos bioativos [Allen T. M. In: Liposomes, New Systems, New Trends in their Applications (F. Puisieux, P. Couvreur, J. Delattre, J.-P. Devissaguet Ed.), Editions de la Sante, Franca, 1995, pp. 125].

Studies in such area showed that different factors affect the circulation half-life of the liposomes-PEG, and ideally, the diameter of the vesicles should be below 200 nm, with a PEG with molecular weight of about 2,000 Da, at a ratio of 3% [Lasic e Martin, Cautela Lipossomas, CRC Press, Inc., Boca Raton, Fla. (1995); Woodle et Col., Biochim. Biophys. Acta 1105:193-200 (1992); Litzinger et Col., Biochim. Biophys. Acta 1190:99-107 (1994); Bedu Addo et Col., Pharm. Res. 13:718-724 (1996)].

Active targeting involves changing of liposomes through their association with a ligand such as a monoclonal antibody, sugar, glycolipid, protein, polymer or changing the composition or size of liposomes for targeting to different organs and cells from the sites where conventional liposomes are accumulated.

Liposomes-based carriers have been proposed for a range of pharmacologically active substances, including antibiotics, hormones and anti-tumor agents [Medical applications of liposomes (D. D. Lasic, D. Papahadjopoulos Ed.), Elsevier Science B. V., Holanda, 1998].

Ang-(1-7) and analogs thereof have great potential for controlling intraocular pressure by regulating local arterial pressure. Another major aspect related to RAS is related to the clear need for an enlargement of the knowledge of its physiological actions, which can provide the development of new therapeutic strategies. However, the conventional way of administration of most anti-hypertensive drugs and specially of biologically active peptides, such as angiotensins and derivatives thereof, suffers from limitations due to its short half-life and when it is sought to obtain information about its chronic actions.

By searching the scientific and patent literature in prior art, the following documents have been found dealing on the subject matter:

U.S. Pat. No. 4,598,070 discloses the obtainment of inclusion compounds between Tripudie (anti-hypertensive) and cyclodextrins (α-ciclodextrina and β-ciclodextrina). Tripamide is slightly soluble in water, therefore the use of cyclodextrins enabled that more soluble compounds could be obtained.

U.S. Pat. No. 5,519,012 discloses an inclusion compound of 1,4-dihydropyridine, anti-hypertensive agent, with methyl-β-cyclodextrin and other derivatives such as hydroxylated β-cyclodextrin. However, this document does not solve the technical issue of administration in the conventional way for hypertensive drugs.

U.S. Pat. No. 4,666,705 discloses a controlled release drugs for hypertension in the form of tablets containing Captopril, ACE inhibitor, along with the polyvinylpyrrolidone (PVP) polymer. The outcome obtained was the increase of residence time of the drug in the body for a period of 4 to 16 hours, still a very short period when compared to the present invention.

Thus, from the researched literature, no documents were found that anticipate or suggest the teachings of the present invention, so that the solution proposed herein has novelty and inventive activity in front of the prior art.

Therefore, there is a clear need for new pharmaceutical compositions that allow for the increase in bioavailability, the duration and intensity of its biological effects.

### SUMMARY OF THE INVENTION

Thus, the present invention has as its objective to solve the sustained problems in the state of the art by the preparation of a pharmaceutical composition using liposomes, cyclodextrins, biodegradable polymers and/or mixtures thereof as a biologically active peptide release system of SEQ ID NO: 1 and derivatives thereof.

The main advantage of this invention is related to the use of biologically active peptide of SEQ ID NO: 1 and derivatives thereof, which has a great potential for controlling intraocular pressure by regulating local arterial pressure, in a conventional way, orally, intravitreous or intraocular injections, or through topical use, e.g. eye drops.

As a first object, the present invention discloses a pharmaceutical composition comprising:
at least one peptide comprising the amino acid sequence with at least 80% similarity or identity with SEQ ID NO: 1; and
controlled release system comprising:
   at least one cyclodextrin or a natural polymer or a modified biopolymer or liposomes or mixture thereof.

As a second object, the present invention discloses a process for producing said pharmaceutical composition comprising the following steps:
encapsulation of the peptide comprising sequence with at least 80% similarity or identity with SEQ ID NO: 1, or
formation of inclusion compound.

As a third object, the present invention discloses a use of a peptide comprising amino acid sequence with at least 80% similarity or identity with SEQ ID NO: 1 in the preparation of a pharmaceutical composition for the treatment of intraocular hypertension or glaucoma-associated diseases.

As a fourth object, the present invention discloses a method of treating intraocular hypertension or glaucoma-associated diseases comprising administering a pharmaceutical composition in a subject in the conventional way of administration.

Further, the inventive concept common to all of the claimed protection contexts is the pharmaceutical composition of a biologically active peptide or analogs for intraocular hypertension or glaucoma bound to a controlled release system consisting of liposomes, cyclodextrins or polymers solving problems related to bioavailability, duration and intensity of their biological effects.

These and other objects of the invention will be readily appreciated by those skilled in the art and to businesses interested in such segment, and will be described in sufficient detail for reproduction thereof in the following description.

### DETAILED DESCRIPTION OF THE INVENTION

The present invention describes a pharmaceutical composition of a biologically active peptide using cyclodextrins and derivatives thereof, liposomes and biodegradable polymers and/or mixtures of such systems as a release system for the purpose of increasing bioavailability, duration and intensity of the biological effects of the peptide. The present invention further describes the preparation and use of said composition.

As a first object, the present invention discloses a pharmaceutical composition comprising:
at least one amino acid sequence with at least 80% similarity or identity with SEQ ID NO: 1; and
controlled release system comprising:
   at least one cyclodextrin or a natural polymer or a modified biopolymer or liposomes or mixture thereof.

In one embodiment of the pharmaceutical composition, the peptide comprises the amino acid sequence of SEQ ID NO: 1

In one embodiment of the pharmaceutical composition, the peptide consists of the amino acid sequence of SEQ ID NO: 1

In one embodiment, the pharmaceutical composition further comprises at least one pharmaceutically acceptable excipient selected from the group consisting of pharmaceutically acceptable carriers, pharmaceutically acceptable additives or combinations thereof.

In one embodiment of the pharmaceutical composition, the pharmaceutically acceptable carrier is selected from the group comprising: water, saline solution, phosphate buffered solutions, and Ringer's solution, dextrose solution, Hank's solution, biocompatible saline solutions containing or not polyethylene glycol, fixed oils, seed oil, ethyl-oleate, or triglyceride.

In one embodiment of the pharmaceutical composition, the additive is selected from the group comprising sodium carboxymethylcellulose, sorbitol, dextran, phosphate buffer, bicarbonate buffer, Tris thimerosal buffer, m-cresol or o-cresol, formalin and benzyl alcohol.

In one embodiment of the pharmaceutical composition, the controlled release system is in the form of capsules, microcapsules, nanocapsules, micro-particles or nano-particles.

In one embodiment of the pharmaceutical composition, the controlled release system comprises liposomes of lipid moiety selected from the group comprising phosphatidylcholine, phosphatidyl serine, phosphatidylglycerol, cardiolipin, cholesterol, phosphatidic acid, sphingolipids, glycolipids, fatty acids, sterols, phosphatidylethanolamine, phospholipids.

In one embodiment of the pharmaceutical composition, the lipid moiety consists of distearoyl-phosphatidylcholine, cholesterol and distearoyl-phosphatidylethanolamine-polyethylene glycol.

In one embodiment of the pharmaceutical composition, the lipid moiety comprises a molar ratio of 4:3:0.2 and 6:5:0.5 distearoyl-phosphatidylcholine:cholesterol:distearoyl-phosphatidylethanolamine-polyethylene glycol.

In one embodiment of the pharmaceutical composition, the lipid moiety comprises a molar ratio of 5:4:0.3 distearoyl-phosphatidylcholine:cholesterol:distearoyl-phosphatidylethanolamine-polyethylene glycol.

In one embodiment of the pharmaceutical composition, the peptide/lipid moiety ratio comprises between 0.01 (w/w) and 0.06 (w/w) and the mean diameter of the vesicles comprises between 0.1 μm and 0.5 μm.

In one embodiment of the pharmaceutical composition, the controlled release system comprises polymer microspheres selected from the group comprising poly (2-hydroxy-ethylmethacrylate), polyacrylamide, lactic acid-based polymers (PLA), polymers based on glycolic acid (PGA), copolymers of lactic and glycolic acid, (PLGA), poly (anhydrides) polymers such as sebacic acid-based polymers PSA and copolymers with hydrophobic polymers.

In one embodiment of the pharmaceutical composition, the microsphere comprises lactic and glycolic acid co-polymers.

In one embodiment of the pharmaceutical composition, the microsphere comprises lactic and glycolic acid co-polymers (PLGA 50:50 w/w).

In one embodiment of the pharmaceutical composition, the peptide/microsphere ratio comprises between 0.01 (w/w) and 0.06 (w/w).

In one embodiment of the pharmaceutical composition, the cyclodextrin is β-cyclodextrin.

As a second object, the present invention discloses a process for the production of said pharmaceutical composition comprising the following steps:
encapsulation of the peptide comprising sequence with at least 80% similarity or identity of SEQ ID NO: 1, or
formation of inclusion compound.

In one embodiment of the process, the encapsulation comprises the following steps:
sterically stabilized liposomes
extrusion of the DRV suspension

In one embodiment of the process, the extrusion of the DRV suspension comprises 200 nm pore polycarbonate membranes.

In one embodiment of the process, the encapsulation comprises the following steps:
Multiple W/O/W emulsion of microspheres
solvent evaporation.

In one embodiment of the process, encapsulation comprises between 10 and 50% efficiency.

In one embodiment of the process, the formation of the inclusion compound comprises the following steps:
mixture of cyclodextrin and peptide solutions
continuous stirring until cyclodextrin dissolution
lyophilization of the mixture

As a third object, the present invention discloses a use of a peptide comprising an amino acid sequence with at least 80% similarity or identity with SEQ ID NO: 1 in the preparation of a pharmaceutical composition for the treatment of diseases associated with intraocular hypertension or glaucoma.

In one embodiment of use, the pharmaceutical composition is in the preparation of a medication for the treatment of diseases associated with intraocular hypertension or glaucoma.

As a fourth object, the present invention discloses a method for treating diseases associated with intraocular hypertension or glaucoma comprising administering said pharmaceutical composition in a subject.

In one embodiment of the method of treatment, the release of the peptide in physiological conditions comprises between 50 and 70% in 8 hours and comprises between 80 and 95% in 48 hours.

The principal advantage of this invention is related to the use of the biologically active peptide of SEQ ID NO: 1 and analogs thereof, which has a great potential for controlling intraocular pressure by regulating local arterial pressure, in a conventional way, orally or by eye drops.

### EXAMPLES—EMBODIMENTS

The examples set forth herein are meant to exemplify one of numerous ways of carrying out the invention, but do not limit the scope thereof.

### Example 1. Preparation of the Peptide of SEQ ID NO: 1 in Liposomes

This example describes the preparation of the peptide of SEQ ID NO: 1 in encapsulated form in sterically stabilized liposomes and improving the bioavailability of the peptide of SEQ ID NO: 1 when administered in this form.

The preparation of peptide of SEQ ID NO: 1 in encapsulated form in liposomes was carried out according to the Kirby and Gregoriadis Method [Biotechnology 2: 979-984, 1984] and followed by extrusion of the DRV suspension (acronym in English for “dehydration-rehydration vesicles”, through 200 nm diameter pore polycarbonate membranes [Nayar et al. Biochim. Biophys. Acta. 986:200-206 (1989)]. Liposomes containing encapsulated peptide were separated from the non-encapsulated peptide by means of dialysis and were sterilized by filtration through 0.22 micrometer sterile membranes. A lipid composition of distearoyl-phosphatidylcholine, cholesterol and distearoyl-phosphatidylethanolamine-polyethylene glycol (MW 2,000) and a molar ratio of 5:4:0.3 were chosen. The amount of encapsulated peptide was determined using the intrinsic fluorescence of SEQ ID NO: 1. Encapsulation efficiency was 12% and a peptide/lipid ratio of 0.03 (w/w). The size of liposomes was determined by quasi-elastic light scattering technique. The mean diameter of the vesicles was 0.19 micrometers. Additionally, the present invention can be optimized for up to 50% encapsulation efficiency.

Liposomes containing SEQ ID NO: 1 (Lang) were unilaterally micro-injected (35 ng of Ang-(1-7) in 200 nL) in the rostro-ventrolateral bulb (RVLB) with a needle (30G) which was slowly inserted into the brain tissue by the dorsal surface using the stereotaxis coordinates: 1.8 mm anterior, 1.8 mm lateral to obex, and only on the pia mater. Empty liposomes (Lvaz) were micro-injected similarly at the same dose of lipid. The arterial pressure was recorded through telemetry for 10 seconds, every 10 minutes, starting 4 previous days and ending 12 days later, in non-disturbed rats with freedom of movement.

Microinjection of Lang produced a significant pressor effect during the daytime period which was maintained for five days. The highest mean arterial pressure (MAP) was obtained on day 3 (114±4 mmHg) which was significantly different from that recorded on day 0 (100±3 mmHg). As expected, Lvaz did not produce significant change in MAP (94±5 mmHg on day 3 vs 90±5 mmHg on day 0). Furthermore, daytime MAP was significantly higher in Lang group than in Lvaz group on days 1, 2, and 3. Night MAP, in contrast to daytime MAP, was not affected significantly by Lang micro-injection.

Previous studies have established that microinjection of Free (non-encapsulated) SEQ ID NO: 1 in the RVLB, at a similar dose (25-50 ng), produces an increase of 15 mmHg for approximately 10 min. The short duration of this effect was assigned to increased metabolism of peptide in vivo.

Therefore, the present technology is characterized by allowing to establish, in chronic conditions, the pressor effect of SEQ ID NO: 1 at RVLB level. It is further characterized by the ability to increase the bioavailability of peptide.

### Example 2.—Preparation of Peptide of SEQ ID NO: 1 in PLGA Microspheres

This example describes the preparation of peptide of SEQ ID NO: 1 in PLGA microspheres and the sustained release of the peptide from the resulting formulation.

Polymer particles were prepared from lactic and glycolic acid co-polymers (PLGA 50:50), by the method of the multiple W/O/W emulsion with later evaporation of the solvent [Jeffery et al. Int. J. Pharm. 77:169-175 (1991)]. Such a method was employed for the encapsulation of Ang-(1-7) with the following steps: 100 mg of PLGA polymer (50:50 w/w) was dissolved in 1 mL of dichloromethane. Next, 1.8 mg of SEQ ID NO: 1 was added, previously dissolved in 200 μl of deionized water, and the mixture was undergone to sonication for obtaining a water/oil (W/O) emulsion. The resulting W/O emulsion was added to 50 mL of a 1% PVA solution (w/v) in deionized water. The mixture was undergone to sonication (5000 revolutions/minute) for approximately 1 minute. Thus, the second water/oil/water emulsion (W/O/W) is formed. The emulsion was maintained at continuous stirring for 2 hours at room temperature for evaporating dichloromethane. Next, formed microspheres were subjected to 3 centrifugation/wash cycles with deionized water. The microspheres were then lyophilized and stored at −20° C.

To determine the amount of incorporated peptide, the peptide was extracted from the polymer particles after polymer dissolution in dichloromethane. The dosage of peptide was carried out by radioimmunoassay [Neves et al., Biochem. Pharmacol. 50:1451-1459 (1995)]. The incorporated amount was 1.9 mg of peptide per g of microspheres, representing a 15% incorporation percentage.

The kinetics of peptide release was evaluated after re-suspending the microspheres in buffered saline solution (pH 7.2) and incubation at 37° C. These experimental conditions represent model physiological conditions. The released peptide was dosed by radioimmunoassay at intervals of 8 hours, 24 hours, and 48 hours. The percentage of peptide released from the microspheres at standard physiological conditions was about 60% in 8 hours, and about 90% at 48 hours.

Therefore, this example illustrates the ability of polymeric microspheres to incorporate the peptide and promote an extended release of the peptide.

### Example 3. Preparation of the Inclusion Compound of β-Cyclodextrin and Derivatives Thereof, and the Peptide of SEQ ID NO: 1

The preparation is made in equimolar ratios of β-cyclodextrin and derivatives thereof, and SEQ ID NO: 1 and/or analogs in aqueous solutions. The mixture of solutions is subjected to continuous stirring until full dissolution of the β-cyclodextrin.

Thereafter the mixture is frozen at liquid nitrogen temperature and subjected to the lyophilization process for 24 hours. Solid, thus obtained, was characterized by physico-chemical analysis techniques. The art that provided major features of the host:guest interaction was the fluorescence and absorption spectroscopy in the ultraviolet-visible region.

The absorption and biological stability tests were performed with solutions of the peptide-cyclodextrin inclusion compound. To carry out the experiments, 12 normal Wistar rats were used, which had previously cannulated the left femoral artery. Animals were divided into 3 experimental groups and subjected to gavage using saline solution (0.9%/50 μL)), SEQ ID NO: 1 (10 μg/50 μl)) and SEQ ID NO: 1 βCD (10 μg/50 μL). Four blood draws (1 mL) were carried out, being the first prior to gavage, and the three others within 2, 6 and 24 hours after gavage

The obtained results demonstrated that SEQ ID NO: 1 βCD is largely absorbed in the TGI, reaching its maximum blood concentration about 6 hours (620±194 pg/mL), returning to near basal values following 24 hours of the gavage (30±8 pg/mL vs 25±10 prior to gavage). Administration of SEQ ID NO: 1 alone also increased the plasma concentration of this peptide 6 hours following its administration (86±13 pg/ml) but this increase was about 8 times less than observed with SEQ ID NO: 1 βcyclodextrin. Administration of saline did not alter the plasma levels of SEQ ID NO: 1. These results show that SEQ ID NO:1 β cyclodextrin may be used for administering SEQ ID NO:1 and likely analogs thereof orally.

### Example 4—Stability Study of Peptide of SEQ ID NO: 1

The results obtained during the Long-Term and accelerated stability study demonstrate that the raw material is stable for 36 months in condition of 5° C.±3° C. and for 6 months in condition of 25° C.±2° C. with 60% relative humidity Example 5—Residual solvents in peptide of SEQ ID NO:1

In the manufacture of pharmaceutical formulations and the chemical synthesis of excipients and drugs, the use of a high number of organic solvents is required, which are not always completely removed during manufacturing processes. These solvents in addition to have no therapeutic value, represent a risk of toxicity to the consumer and carry with them possible adverse effects, making its analysis essential. The ideal is that the presence of these undesirable solvents is the smallest possible (RDC Resolution #57, 2009; United States Pharmacopeia, 2009; International Conference on Harmonization, 1997).

The test of residual solvents is performed to evaluate the amount of organic solvent present in a given formulation and to verify whether this product has the concentration allowed by law. These tests are generally not mentioned in specific monographs, since the solvents employed vary from one manufacturer to the other. (United States Pharmacopeia, 2009).

According to developed methodology, it has been found that solvents used in the synthesis pathway, Dimethylformamide, Methanol, Acetonitrile, Dichloromethane, Diethylether, Acetic Acid and Trifluoroacetic acid are controlled by the manufacturer through techniques developed by GC (for dimethylformamide, Methanol, Acetonitrile, Dichloromethane, Diethylether solvents) and HPLC for Acetic Acid and Trifluoroacetic Acid solvents. They assume specifically the values according to table 2:

| Solvent | Source | Specification Limit |
|---|---|---|
| Dimethylformamide | Reactive Solvent | 880 ppm |
| Methanol | Reactive Solvent | 3000 ppm |
| Acetonitrile | Reactive Solvent | 410 ppm |
| Dichloromethane | Reactive Solvent | 600 ppm |
| Diethylether | Reactive Solvent | 600 ppm |
| Acetic Acid | Reactive Solvent | It determines |
| | | and reports |
| Trifluoroacetic Acid | Reactive Solvent | 10000 ppm |

### Example 6—Physical-Chemical Tests of Peptide of SEQ ID NO: 1

To ensure the quality of the active substance, in addition to the tests mentioned above, the following tests were proposed for evaluating raw material quality: mass spectrometry in order to characterize the molecule, quantification of amino acids content of liquid peptide and peptide content (HPLC), appearance of sample, solubility, content (purity), water content (HPLC), and microbiological tests.

### Example 7—Stability Study of Peptide of SEQ ID NO: 1

Stability is defined as the time during which pharmaceutical expertise or even raw material considered alone is maintained within specified limits and throughout the period of storage and use, the same conditions and characteristics that had upon the time of their manufacture. It can also be defined as the time period comprised between the time at which the product is being manufactured to that when its potency is reduced to not more than 10%, since the alteration products are all securely identified and previously recognized their effects (Taboranski, 2003; Vehabovic et al. 2003; Stulzer & Silva, 2006).

Stability study was carried out in two conditions, the condition A being at 5° C.±3° C. with no humidity and condition B 25° C.±2° C. with the relative humidity of 60%±5%. The study demonstrated that for all tests that the methodology proposes and from time 0 to time of 36 months in the long-term condition and was also stable in the accelerated condition for 6 months.

Therefore, the present technology, based on the association of the peptide to the cyclodextrin, allows to increase the peptide bioavailability orally, as well as in the form of intravitreal or intraocular injection and/or by topical use, for example, eye drops.

Additional tests were performed in order to demonstrate that excellent results were obtained in the association of peptide in the mixture of cyclodextrins, polymers and liposomes.

Those skilled in the art will appreciate the knowledge presented herein and may reproduce the invention in the disclosed embodiments and in other variants, encompassed within the scope of the appended claims.

1. Pharmaceutical composition comprising:
at least one peptide comprising the amino acid sequence with at least 80% similarity or identity with SEQ ID NO: 1; and
controlled release system comprising:
at least one cyclodextrin or a natural polymer or a modified biopolymer or liposomes or mixture thereof.

2. Pharmaceutical composition according to claim 1, wherein the peptide comprises the amino acid sequence as defined in SEQ ID NO: 1.

3. Pharmaceutical composition according to claim 1, wherein the peptide consists the amino acid sequence as defined in SEQ ID NO: 1.

4. Pharmaceutical composition according to claim 1, wherein the composition further comprises at least one pharmaceutically acceptable excipient selected from the group consisting of pharmaceutically acceptable carriers, pharmaceutically acceptable additives or combinations thereof.

5. Pharmaceutical composition according to claim 4, wherein the pharmaceutically acceptable carrier is selected from the group comprising: water, saline solution, phosphate buffered solutions, a Ringer's solution, dextrose solution, Hank's solution, biocompatible saline solutions containing or not polyethylene glycol, fixed oils, seed oil, ethyl-oleate, or triglyceride.

6. Pharmaceutical composition according to claim 4, wherein the additive is selected from the group comprising sodium carboxymethylcellulose, sorbitol, dextran, phosphate buffer, bicarbonate buffer, Tris thimerosal buffer, m-cresol or o-cresol, formalin and benzyl alcohol.

7. Pharmaceutical composition according to claim 1, wherein the controlled release system is in the form of capsules, microcapsules, nanocapsules, micro-particles or nano-particles.

8. Pharmaceutical composition according to claim 1, wherein the controlled release system comprises liposomes of lipid moiety selected from the group comprising phosphatidylcholine, phosphatidylserine, phosphatidylglycerol, cardiolipin, cholesterol, phosphatidic acid, sphingolipids, glycolipids, fatty acids, sterols, phosphatidylethanolamine, phospholipids.

9. Pharmaceutical composition according to claim 8, wherein the lipid moiety consists of distearoyl-phosphatidylcholine, cholesterol and distearoyl-phosphatidylethanolamine-polyethylene glycol.

10. Pharmaceutical composition according to claim 9, wherein the lipid moiety comprises a molar ratio of 4:3:0.2 to 6:5:0.5 of distearoyl-phosphatidylcholine:cholesterol:distearoyl-phosphatidylethanolamine-polyethylene glycol.

11. Pharmaceutical composition according to claim 10, wherein the lipid moiety comprises a molar ratio of 5:4:0.3 of distearoyl-phosphatidylcholine:cholesterol:distearoyl-phosphatidylethanolamine-polyethylene glycol.

12. Pharmaceutical composition according to claim 8, wherein the peptide/lipid moiety ratio comprises 0.01 (w/w) to 0.06 (w/w) and the mean diameter of the vesicles comprises between 0.1 μm to 0.5 μm.

13. Pharmaceutical composition according to claim 1, wherein the controlled release system comprises polymer microspheres selected from the group comprising poly (2-hydroxy-ethylmethacrylate)), polyacrylamide, lactic acid-based polymers (PLA), polymers based on glycolic acid (PGA), copolymers of lactic and glycolic acid, (PLGA), poly(anhydrides) polymers such as sebacic acid-based polymers PSA and copolymers with hydrophobic polymers.

14. Pharmaceutical composition according to claim 13, wherein the microsphere comprises lactic and glycolic acid co-polymers.

15. Pharmaceutical composition according to claim 13, wherein the microsphere comprises lactic and glycolic acid co-polymers (PLGA 50:50 w/w).

16. Pharmaceutical composition according to claim 13, wherein the peptide/microsphere ratio comprises between 0.01 (w/w) to 0.06 (w/w).

17. Pharmaceutical composition according to claim 1, wherein the cyclodextrin is beta-cyclodextrin.

18. Process for producing the pharmaceutical composition as defined in claim 1, comprising the following steps:
encapsulation of the peptide comprising sequence with at least 80% similarity or identity with SEQ ID NO: 1, or
formation of inclusion compound.

19. Process according to claim 18, wherein the encapsulation comprises the following steps:
sterically stabilized liposomes;
extrusion of DRV suspension.

20. Process according to claim 19, wherein the extrusion of DRV suspension comprises 200 nm pore polycarbonate membranes.

21. Process according to claim 19, wherein the encapsulation comprises the following steps:
multiple W/O/W emulsion of microspheres;
solvent evaporation.

22. Process according to claim 18, wherein the encapsulation comprises between 10 and 50% efficiency.

23. Process according to claim 18, wherein the formation of inclusion compound comprises the following steps:
mixture of cyclodextrin and peptide solutions;
continuous stirring until cyclodextrin dissolution;
lyophilization of the mixture.

24. Use of a peptide comprising an amino acid sequence with at least 80% similarity or identity with SEQ ID NO:1, for the preparation of a pharmaceutical composition for the treatment of diseases associated with intraocular hypertension or glaucoma.

25. Use of a pharmaceutical composition as defined in claim 1, for the preparation of a medicament for the treatment of diseases associated with intraocular hypertension or glaucoma.

26. Method for treating diseases associated with intraocular hypertension or glaucoma, comprising administering a pharmaceutical composition, as defined in claim 1, in a subject.

27. Method for treating diseases associated with intraocular hypertension or glaucoma, comprising administering a pharmaceutical composition obtained by the process, as defined in claim 19, wherein the release of the peptide in physiological conditions comprises between 50 and 70% in 8 hours and comprising between 80 and 95% in 48 hours.
